(19) United States
(12) Patent Application Publication
(10) Pub. No.: US 2025/0275628 A1
(43) Pub. Date: Sep. 4, 2025

(54) WRIST REST TYPE PHYSICAL THERAPY DEVICE

(71) Applicant: SHENZHEN RUIXINCHENG TECHNOLOGY CO., LTD, Shenzhen (CN)

(72) Inventor: Yuze He, Shenzhen (CN)

(21) Appl. No.: 19/207,045

(22) Filed: May 13, 2025

(51) Int. Cl. A47B 21/03; A61N 5/06

(52) U.S. Cl. CPC A47B 21/0371; A61N 5/0613; A61N 2005/0643

(57) ABSTRACT

The present disclosure provides a wrist rest type physical therapy device. The wrist rest type physical therapy device includes: a housing assembly, a physical therapy lamp set and a flexible supporting pad, in which, the housing assembly includes a lamp set mounting chamber, and at least part of the physical therapy lamp set is arranged in the lamp set mounting chamber; the flexible supporting pad is arranged on the housing assembly and used for supporting a part to be subjected to physical therapy; the physical therapy lamp set is used for emitting physical therapy light to the flexible supporting pad so as to irradiate the part to be subjected to physical therapy. Therefore, the whole wrist rest type physical therapy device can support and hold the part to be subjected to physical therapy of a user and can also conduct physical therapy with the physical therapy light.

### FIELD OF THE INVENTION

The present invention relates to the technical field of wrist rests, and particularly relates to a wrist rest type physical therapy device.

### BACKGROUND OF THE INVENTION

In behaviors such as playing ball and working, people use the wrist frequently, resulting in fatigue and injury of the wrist. People need wrist rests when working in the office.

Existing wrist rests can only play a passive supporting role for the user through its shape to relieve fatigue, and they have simple function.

### SUMMARY OF THE INVENTION

In view of this, the present disclosure provides a wrist rest type physical therapy device.

The present disclosure provides a wrist rest type physical therapy device, including:
a housing assembly comprising a lamp set mounting chamber;
a physical therapy lamp set of which at least part is arranged in the lamp set mounting chamber; and
a flexible supporting pad which is arranged on the housing assembly and used for supporting a part to be subjected to physical therapy; and
the physical therapy lamp set is used for emitting physical therapy light to the flexible supporting pad so as to irradiate the part to be subjected to physical therapy.

As an embodiment of the present invention, the housing assembly includes a bottom cover and a base arranged on the bottom cover, and the base and the bottom cover define the lamp set mounting chamber.

As an embodiment of the present invention, the physical therapy lamp set includes a PCB, lamp bead members arranged on the PCB, a light guide member arranged corresponding to the lamp bead members; and
the lamp bead members are used for emitting the physical therapy light through the light guide member.

As an embodiment of the present invention, the base further includes first light holes which are in communication with the lamp set mounting chamber, and the first light holes are arranged corresponding to the light guide member.

As an embodiment of the present invention, the light guide member includes a fixing plate, and light guide columns arranged between the fixing plate and the lamp bead members; and
a plurality of lamp bead members are provided in correspondence to the light guide columns; and/or
the fixing plate is fixed to the PCB by a fixing member.

As an embodiment of the present invention, the base includes a plate body and a boss arranged on the plate body, and a hollow groove is formed in the side of the boss facing the bottom cover;
the hollow groove and the bottom cover form the lamp set mounting chamber; and
the boss comprises an annular plate arranged on the plate body and a top plate arranged on the annular plate far away from the bottom cover, and the top plate comprises the first light holes.

As an embodiment of the present invention, the physical therapy lamp set further includes a touch key which is arranged on the fixing plate and is electrically connected to the PCB, and the top plate further includes a first key hole which is in communication with the lamp set mounting chamber; and
the touch key protrudes out of the first key hole; and/or
a plurality of first light holes are arranged around the first key hole.

As an embodiment of the present invention, a boss groove is formed in the side of the flexible supporting pad facing the base;
the flexible supporting pad is connected to the bottom cover, and the boss groove is arranged corresponding to the boss.

As an embodiment of the present invention, a sunken hand supporting portion is formed on the side of the flexible supporting pad away from the base; and/or
the flexible supporting pad further includes second light holes which are formed in the side of the boss groove away from the base and are in communication with the boss groove, and the second light holes are arranged corresponding to the first light holes; and/or
the flexible supporting pad further includes a second key hole which is formed in the side of the boss groove away from the base and is in communication with the boss groove; and the touch key protrudes out of or is flush with the surface of the side of the flexible supporting pad away from the base through the second key hole.

As an embodiment of the present invention, the wrist rest type physical therapy device further includes a flexible wrapping portion which sleeves the flexible supporting pad.

Beneficial effects: according to the wrist rest type physical therapy device provided by the present disclosure, a lamp set mounting chamber is formed in a housing assembly, at least part of a physical therapy lamp set is arranged in the lamp set mounting chamber, and then a flexible supporting pad is arranged on the housing assembly to support the hands/feet and other body parts of a user; moreover, physical therapy light is emitted to the flexible supporting pad by the physical therapy lamp set, thus parts to be subjected to physical therapy, such as the hands of the user, can be supported and lifted, and furthermore, the physical therapy light can be further used for carrying out physical therapy on the parts to be subjected to physical therapy of the user, thereby effectively relieving the fatigue of the hands and other parts of the user, and repairing the injury; and moreover, the whole structure is simple and convenient to carry, so the whole wrist rest type physical therapy device has good convenience and multifunctionality, the user can also perform physical therapy at home, and the physical therapy cost is effectively reduced.

### BRIEF DESCRIPTION OF THE DRAWINGS

To describe the technical solutions in the embodiments of the present invention more clearly, the following briefly introduces the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a structural schematic diagram of one embodiment of a wrist rest type physical therapy device provided by the present disclosure;

FIG. 2 is a structural schematic diagram of the other embodiment of a wrist rest type physical therapy device provided by the present disclosure;

FIG. 3 is an explosion decomposition schematic diagram of a wrist rest type physical therapy device shown in FIG. 2;

FIG. 4 is a schematic diagram of assembling of a bottom cover and a physical therapy lamp set in a wrist rest type physical therapy device in FIG. 1;

FIG. 5 is a structural schematic diagram of a base in a wrist rest type physical therapy device in FIG. 1;

FIG. 6 is a structural schematic diagram of a flexible supporting pad in a wrist rest type physical therapy device in FIG. 1; and

FIG. 7 is a section schematic diagram of a wrist rest type physical therapy device in FIG. 2.

### DETAILED DESCRIPTION OF SOME EMBODIMENTS

The technical solution in the embodiments of the present disclosure will be clearly and completely described below in conjunction with the accompanying drawings in the embodiments of the present disclosure. Obviously, the described embodiments are partial embodiments of the present disclosure, not all embodiments. Based on the embodiments in the present disclosure, all other embodiments obtained by those skilled in the art without creative work fall within the scope of protection of the present disclosure.

It is to be understood that all directional indications (such as up, down, left, right, front, rear . . . ) in the embodiment of the present disclosure are only used for describing relative position relationship between components in a specific posture, the movement, etc., and if the specific posture changes, the directional indications will change accordingly.

It is also to be understood that when an element is described to be “fixed to” or “arranged on” another element, it can be either directly on the other component or there may be a middle component at the same time. When one element is described to be “connected” to another component, it can be either directly connected to another component or indirectly connected by the middle component.

The terms used in this description of the present disclosure are for the purpose of describing specific embodiments only and are not intended to limit the present disclosure. If the description of “first”, “second”, etc. in this application is for descriptive purposes only and should not be construed as indicating or implying their relative importance or implying the number of technical features indicated. Thus, defining the “first” and “second” features may explicitly or implicitly include at least one of those features.

It is also to be further understood that the term “and/or” used in the description of this application and the accompanying claims of the present disclosure refers to any combination of one or more of the items listed in association and all possible combinations, and includes such combinations.

With reference to FIG. 1 to FIG. 7, the present disclosure provides a wrist rest type physical therapy device 10, which includes a housing assembly 100, a physical therapy lamp set 200 and a flexible supporting pad 300.

As shown in FIG. 7, the housing assembly 100 includes a lamp set mounting chamber 110, and at least part of the physical therapy lamp set 200 is arranged in the lamp set mounting chamber 110. The flexible supporting pad 300 is arranged on the housing assembly 100 and used for supporting a part to be subjected to physical therapy, specifically, such as hands/feet.

Optionally, the physical therapy lamp set 200 is used for emitting physical therapy light to the flexible supporting pad 300, specifically, the physical therapy light can be red light and near-red light with preset wavelength, and the physical therapy light can irradiate the part to be subjected to physical therapy, such as the hands/feet of a user, so that physical therapy can be performed on the user with the part to be subjected to physical therapy on the flexible supporting pad. It can help to alleviate user fatigue, etc.

Optionally, the physical therapy light can pass through the housing assembly and the flexible supporting pad to directly irradiate the part to be subjected to physical therapy of the user.

Optionally, the physical therapy light can also be certainly blocked by the flexible supporting pad, but some of it will irradiate the part to be subjected to physical therapy of the user, which is not limited here.

Optionally, the part to be subjected to physical therapy can be specifically the wrist, fingers, palms, arms, ankles, etc., which is not limited here. In an optional embodiment, the physical therapy light used in the present disclosure may be specifically a kind of red light (the wavelength is usually greater than or equal to 600 nm, less than or equal to 700 nm, specifically, it may be 600 nm, 630 nm, 660 nm, 700 nm, etc.). Human tissues are irradiated with the red light, which can penetrate to a depth of about 1-5 mm (deep into the dermis and partly into the subcutaneous tissue). Therefore, it can irradiate human body parts with at least one of the following functions:
1, promoting skin repair and collagen synthesis (anti-aging and wound healing).
2, reducing inflammation (such as acne and dermatitis).
3, improving mitochondrial function (by activating cytochrome C oxidase).

In an optional embodiment, the physical therapy light used in the present disclosure may be a near-red light (the wavelength is usually greater than 700 nm, less than or equal to 1100 nm, specifically, it may be 701 nm, 830 nm, 850 nm, 980 nm). Human tissues are irradiated with the near-red light, which can penetrate to a depth of about 5-10 cm (deep into muscle, bone and nerve tissue). Therefore, it can irradiate human body parts with at least one of the following functions:
1, deeply repairing tissue (muscle damage, joint pain).
2, regenerating nerve regeneration (e.g., peripheral neuropathy).
3, resisting inflammation and easing pain (by inhibiting pro-inflammatory factors).

In the above embodiment, the wrist rest type physical therapy device 10 is provided, the lamp set mounting chamber 110 is formed in the housing assembly 100, at least part of the physical therapy lamp set 200 is arranged in the lamp set mounting chamber 110, and then the flexible supporting pad 300 is arranged on the housing assembly 100 to support the hands/feet and other body parts of the user; moreover, physical therapy light is emitted to the flexible supporting pad by the physical therapy lamp set, thus parts to be subjected to physical therapy, such as the hands of the user, can be supported and lifted, and furthermore, the physical therapy light can be further used for carrying out physical therapy on the user, thereby effectively relieving the fatigue of the hands and other parts of the user, and repairing the injury; and moreover, the whole structure is simple and convenient to carry, so the whole wrist rest type physical therapy device 10 has good convenience and multifunctionality, the user can also perform physical therapy at home, and the physical therapy cost is effectively reduced.

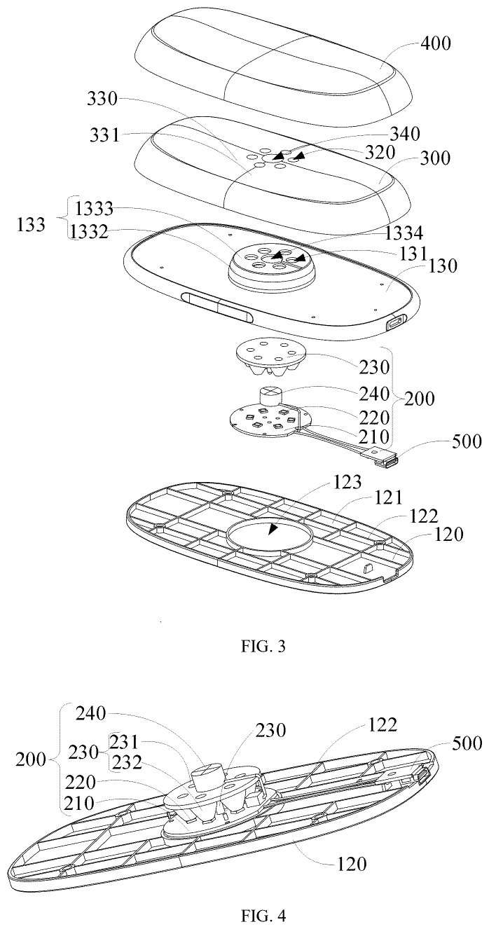

As shown in FIG. 3 and FIG. 7, the housing assembly 100 includes a bottom cover 120 and a base 130 arranged on the bottom cover 120, and the base 130 and the bottom cover 120 define the lamp set mounting chamber 110.

As shown in FIG. 3 and FIG. 4, the physical therapy lamp set 200 includes a PCB 210, lamp bead members 220 and a light guide member 230, in which, the lamp bead members 220 are arranged on the PCB 210, the light guide member 230 is arranged corresponding to the lamp bead members 220, and the lamp bead members 220 are used for emitting the physical therapy light through the light guide member 230, specifically, the physical therapy light is emitted towards the flexible supporting pad 300 through the light guide member 230.

Optionally, the lamp bead members 220 arranged on the PCB 210 can be directly fixed to the PCB 210, and therefore the size of the whole physical therapy lamp set 200 is effectively reduced; or, the lamp bead members 220 can be electrically connected to the PCB 210, which is not limited here.

As shown in FIG. 3 and FIG. 5, the base 130 further includes first light holes 131 which are in communication with the lamp set mounting chamber 110, and the first light holes 131 are arranged corresponding to the light guide member 230. The first light holes 131 can be through holes, and the physical therapy light emitted by the lamp bead members 220 is emitted towards the flexible supporting pad 300 through the light guide member 230 and the first light holes 131 in sequence.

In an optional embodiment, one lamp bead member 220 may be provided.

In an optional embodiment, a plurality of lamp bead members 220 may be provided, specifically, two, three or more lamp bead members 220 may be provided.

As shown in FIG. 4 and FIG. 7, the light guide member 230 includes a fixing plate 231, and light guide columns 232 arranged between the fixing plate 231 and the lamp bead members 220. Optionally, the fixing plate 231 and the PCB 210 are arranged at an interval, and the light guide columns 232 are fixed to the fixing plate 231 and arranged corresponding to the lamp bead members 220.

Optionally, the fixing plate 231 can be fixed to the PCB 210 by a fixing member 233, such as a threaded member and an adhesive layer.

In an optional embodiment, the fixing plate 231 can also be fixed to the PCB 210 by means of the light guide columns 232, which is not limited here.

In an optional embodiment, a plurality of light guide columns 232 can be provided in correspondence to the lamp bead members 220, that is, each lamp bead member 220 corresponds to one light guide column 232.

A plurality of corresponding first light holes 131 can be provided in correspondence to the light guide columns 232, that is, each light guide column 232 corresponds to one first light hole 131.

In other embodiments, the first light holes 131 can also be annular holes and can correspond to the plurality of light guide columns 232.

Optionally, the light guide columns 232 can conduct light uniformizing, light diffusing and the like on the physical therapy light emitted by the lamp bead members 220 in the light guide process.

As shown in FIG. 3 and FIG. 5, the base 130 includes a plate body 132 and a boss 133 arranged on the plate body 132, a hollow groove 1331 is formed in the side of the boss 133 facing the bottom cover 120, and the hollow groove 1331 and the bottom cover 120 form the lamp set mounting chamber 110.

Optionally, a plurality of first reinforcing ribs 1321 are arranged by crossing in the direction of the plate body 132 facing the bottom cover.

In an optional embodiment, the bottom cover 120 includes a main plate 121, a plurality of second reinforcing ribs 122 are arranged by crossing on the main plate 121, and a mounting groove 123 is further formed in the main plate 121.

Optionally, the base 130 and the bottom cover 120 can be fixed in a threaded mode, a pasting mode and the like, and meanwhile, the hollow groove 1331 and the mounting groove 123 form the lamp set mounting chamber 110 together.

In an optional scene, the PCB 210, the lamp bead members 220 and the light guide member 230 can be mounted on the bottom cover 120 firstly.

Optionally, the PCB 210 can be mounted into the mounting groove 123 in the bottom cover 120, then the base 130 is mounted on the bottom cover 120 and the hollow groove 1331 is mounted corresponding to the mounting groove 123, and therefore at least parts of the PCB 210, the lamp bead members 220 and the light guide member 230 are accommodated in the hollow groove 1331.

Optionally, the PCB 210, the lamp bead members 220 and the light guide member 230 can be all located in the lamp set mounting chamber 110.

In an optional embodiment, the bottom cover 120 and the base 130 are made of but not limited to one or more of hard plastic materials such as plastic, metal and silica gel.

In an optional embodiment, the flexible supporting pad 300 is made of but not limited to one or more of soft materials such as memory foam, gel and silica gel.

In an optional embodiment, the hardness of the bottom cover 120 and the base 130 is greater than that of the flexible supporting pad 300.

In an optional embodiment, the light guide member 230 can be made of one or more of transparent materials such as acrylic, glass, silica gel and PC.

As shown in FIG. 3 and FIG. 5, the boss 133 includes an annular plate 1332 arranged on the plate body 132, and a top plate 1333 arranged on the annular plate 1332 far away from the bottom cover 120, and the top plate 1333 includes the first light holes 131.

Optionally, the annular plate 1332 can be arranged in a conical shape or a columnar shape.

Optionally, the annular plate 1332 can be arranged around and against the light guide member 230, and specifically, the annular plate 1332 can be arranged and against the fixing plate 231.

As shown in FIG. 4 and FIG. 7, the physical therapy lamp set 200 further includes a touch key 240 which is arranged on the fixing plate 231 and is electrically connected to the PCB 210, and the top plate 1333 further includes a first key hole 1334 which is in communication with the lamp set mounting chamber 110.

Optionally, the touch key 240 can also be directly arranged on the PCB 210 and penetrates through the fixing plate 231, which is not limited here.

Optionally, the touch key 240 penetrates through and protrudes out of the first key hole 1334.

Optionally, a plurality of first light holes 131 are arranged around the first key hole 1334.

Optionally, the first light holes 131 are of an annular structure and can be arranged around the first key hole 1334.

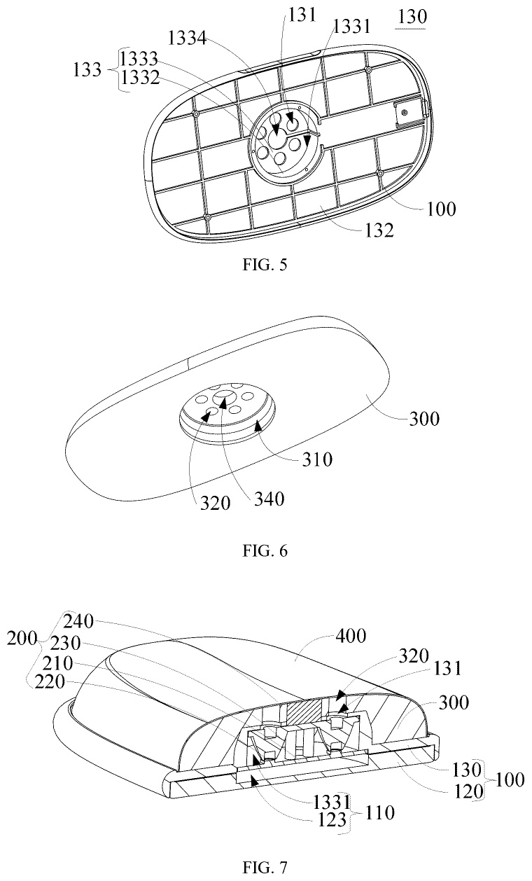

As shown in FIG. 6, a boss groove 310 is formed in the side of the flexible supporting pad 300 facing the base 130, the flexible supporting pad 300 is connected to the bottom cover 120, and the boss groove 310 is arranged corresponding to the boss 133.

Optionally, the flexible supporting pad 300 can be fixed to the base 130 in a bonding manner or threaded manner, and meanwhile, the boss groove 310 is arranged corresponding to the boss 133, namely, at least part of the boss 133 is located in the boss groove 310.

As shown in FIG. 1, a sunken hand supporting portion 330 is formed on the side of the flexible supporting pad 300 away from the base 130, and the sunken hand supporting portion 330 is of a structure with two sides inwards concaved towards the middle so as to fit the hands/feet and other body parts of the user, and thus the hands/feet and other body parts of the user can be stably supported.

Optionally, the flexible supporting pad 300 further includes second light holes 320 which are formed in the side of the boss groove 310 far away from the base 130 and are in communication with the boss groove 310, and the second light holes 320 are arranged corresponding to the first light holes 131.

Optionally, the sunken hand supporting portion 330 further includes a flat laying portion 331 which is located in a middle area of the sunken hand supporting portion 330.

Optionally, the flat laying portion 331 is arranged corresponding to the boss 133

Optionally, the second light holes 320 are formed in the flat laying portion 331.

Optionally, the physical therapy light emitted by the lamp bead members 220 sequentially passes through the light guide columns 232, the first light holes 131 and the second light holes 320 to be emitted.

Optionally, the flexible supporting pad 300 further includes a second key hole 340 which is formed in the side of the boss groove 310 away from the base 130 and is in communication with the boss groove 310; and optionally, the touch key 240 protrudes out of or is flush with the surface of the side of the flexible supporting pad 300 away from the base 130 through the second key hole 340.

Optionally, the second key hole 340 is also located in the flat laying portion 331.

Optionally, the touch key 240 can protrude out of the flat laying portion 331 through the two key holes, so that the touch key 240 is higher than the flat laying portion 331, or the touch key 240 is flush with the flat laying portion 331, which is not limited here.

Optionally, the touch key 240 can be a capacitive sensing switch specifically, and when the user places the hands/feet and other body parts on the sunken hand supporting portion 330, specifically, on the flat laying portion 331, the touch key 240 can sense the situation, and therefore the lamp bead members 220 are turned on to emit the physical therapy light.

In an optional embodiment, the touch key 240 can also be other contact or light sensors, which is not limited here.

Optionally, the boss 133 is arranged, and the boss groove 310 is formed in the flexible supporting pad 300, so that on one hand, the assembling stability of the flexible supporting pad 300 and the base 130 can be improved, and on the other hand, the boss 133 with high hardness is arranged below the flat laying portion 331 of the flexible supporting pad 300, thereby effectively improving the supporting stability for the user, reducing the deformation of the flexible supporting pad 300, and prolonging the service life of the flexible supporting pad 300; and the hollow groove 1331 is formed in the boss 133, it can be matched with the bottom cover 120 to well fix the physical therapy lamp set 200, thus the size of the whole wrist rest type physical therapy device 10 is reduced, and the stability of the whole structure is improved.

In an optional embodiment, the wrist rest type physical therapy device 10 further includes a flexible wrapping portion 400 which sleeves the flexible supporting pad 300.

Optionally, the flexible wrapping portion 400 can be made of milk silk, tencel, leather and other materials, so that the whole support physical therapy device has good comfort.

In an optional embodiment, the thickness of the flexible wrapping portion 400 is less than that of the flexible supporting pad 300, so that the influence of the flexible wrapping portion 400 on penetrating of the physical therapy light is reduced.

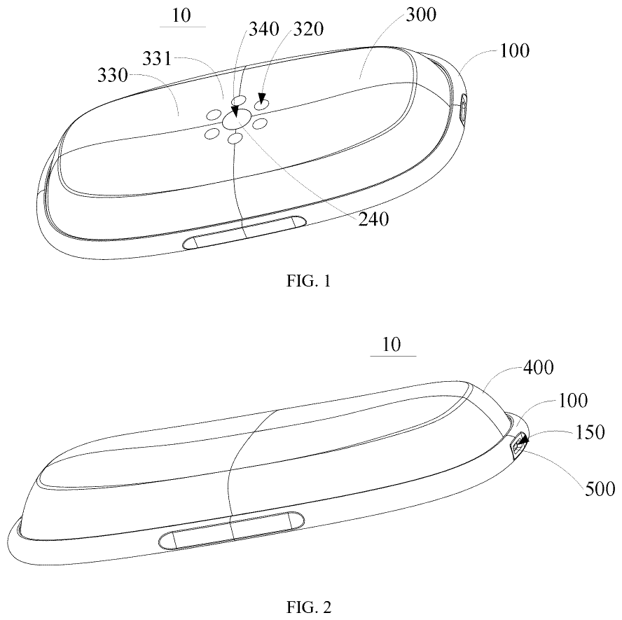

As shown in FIG. 2, the housing assembly 100 further includes a side opening 150, the wrist rest type physical therapy device 10 further includes an electric connection interface 500 arranged on the side opening 150, and the electric connection interface 500 is electrically connected to the PCB 210.

In conclusion, the wrist rest type physical therapy device 10 is provided, the lamp set mounting chamber 110 is formed in the housing assembly 100, at least part of the physical therapy lamp set 200 is arranged in the lamp set mounting chamber 110, and then the flexible supporting pad 300 is arranged on the housing assembly 100 to support the hands/feet and other body parts of the user; moreover, the physical therapy light is emitted to the flexible supporting pad by the physical therapy lamp set, thus the parts to be subjected to physical therapy, such as the hands of the user, can be supported and lifted, and furthermore, the physical therapy light can be further used for carrying out physical therapy on the user, thereby effectively relieving the fatigue of the hands and other parts of the user, and repairing the injury; and moreover, the whole structure is simple and convenient to carry, so the whole wrist rest type physical therapy device 10 has good convenience and multifunctionality, the user can also perform physical therapy at home, and the physical therapy cost is effectively reduced. The physical therapy can be carried out, the whole structure is simple, and therefore, the convenience of physical therapy can be effectively improved, and the cost for physical therapy is decreased.

Without contradicting each other, those skilled in the art may link and combine the different embodiments or examples described in this specification and the features of the different embodiments or examples.

The foregoing is only a specific embodiment of the present disclosure, but the scope of protection of the present disclosure is not limited to this, and any person skilled in the art can easily think of various equivalent modifications or substitutions within the scope of the technology disclosed in the present disclosure, and these modifications or substitutions shall be covered by the scope of protection of the present disclosure. Therefore, the scope of protection of the present disclosure shall be subject to the scope of protection of the claims.

What is claimed is:

1. A wrist rest type physical therapy device, comprising:
a housing assembly comprising a lamp set mounting chamber;
a physical therapy lamp set of which at least part is arranged in the lamp set mounting chamber; and
a flexible supporting pad which is arranged on the housing assembly and used for supporting a part to be subjected to physical therapy; and
the physical therapy lamp set is used for emitting physical therapy light to the flexible supporting pad so as to irradiate the part to be subjected to physical therapy.

2. The wrist rest type physical therapy device according to claim 1, wherein the housing assembly comprises a bottom cover and a base arranged on the bottom cover, and the base and the bottom cover define the lamp set mounting chamber.

3. The wrist rest type physical therapy device according to claim 2, wherein the physical therapy lamp set comprises a PCB, lamp bead members arranged on the PCB, a light guide member arranged corresponding to the lamp bead members; and
the lamp bead members are used for emitting the physical therapy light through the light guide member.

4. The wrist rest type physical therapy device according to claim 3, wherein the base further comprises first light holes which are in communication with the lamp set mounting chamber, and the first light holes are arranged corresponding to the light guide member.

5. The wrist rest type physical therapy device according to claim 4, wherein the light guide member comprises a fixing plate, and light guide columns arranged between the fixing plate and the lamp bead members; and
a plurality of lamp bead members are provided in correspondence to the light guide columns.

6. The wrist rest type physical therapy device according to claim 5, wherein the fixing plate is fixed to the PCB by a fixing member.

7. The wrist rest type physical therapy device according to claim 5, wherein the base comprises a plate body and a boss arranged on the plate body, and a hollow groove is formed in the side of the boss facing the bottom cover;
the hollow groove and the bottom cover form the lamp set mounting chamber; and
the boss comprises an annular plate arranged on the plate body and a top plate arranged on the annular plate far away from the bottom cover, and the top plate comprises the first light holes.

8. The wrist rest type physical therapy device according to claim 7, wherein the physical therapy lamp set further comprises a touch key which is arranged on the fixing plate and is electrically connected to the PCB, and the top plate further comprises a first key hole which is in communication with the lamp set mounting chamber; and
the touch key protrudes out of the first key hole.

9. The wrist rest type physical therapy device according to claim 8, wherein a plurality of first light holes are arranged around the first key hole.

10. The wrist rest type physical therapy device according to claim 8, wherein a boss groove is formed in the side of the flexible supporting pad facing the base; and
the flexible supporting pad is connected to the bottom cover, and the boss groove is arranged corresponding to the boss.

11. The wrist rest type physical therapy device according to claim 10, wherein a sunken hand supporting portion is formed on the side of the flexible supporting pad away from the base.

12. The wrist rest type physical therapy device according to claim 10, wherein the flexible supporting pad further comprises second light holes which are formed in the side of the boss groove away from the base and are in communication with the boss groove, and the second light holes are arranged corresponding to the first light holes.

13. The wrist rest type physical therapy device according to claim 10, wherein the flexible supporting pad further comprises a second key hole which is formed in the side of the boss groove away from the base and is in communication with the boss groove; and the touch key protrudes out of or is flush with the surface of the side of the flexible supporting pad away from the base through the second key hole.
